(19) United States
(12) Patent Application Publication
(10) Pub. No.: US 2025/0137994 A1
(43) Pub. Date: May 1, 2025

(54) MASS SPECTROMETRY METHOD AND SYSTEM

(71) Applicants: UNIVERSITÄTS-KINDERSPITAL BEIDER BASEL, Basel (CH); Jinan University, Guangzhou (CN)

(72) Inventors: Pablo MARTINEZ-LOZANO SINUES, Zürich (CH); Kapil Dev SINGH, Zürich (CH); Kim ARNOLD, Steinhausen (CH); Xue LI, Guangzhou (CN); Xing CHEN, Shanghai (CN)

(73) Assignees: UNIVERSITÄTS-KINDERSPITAL BEIDER BASEL, Basel (CH); Jinan University, Guangzhou (CN)

(21) Appl. No.: 18/836,653

(22) PCT Filed: Feb. 8, 2022
(86) PCT No.: PCT/CN2022/075436

§ 371 Date: Aug. 7, 2024

(51) Int. Cl. G01N 33/497; H01J 49/04; H01J 49/16; H01J 49/42

(52) U.S. Cl. CPC G01N 33/4975; H01J 49/0422; H01J 49/165; H01J 49/425; G01N 2458/15

(57) ABSTRACT

In a mass spectrometry method, a deuterium-resolved mass spectrum of a gas sample comprising volatile metabolites that have been excreted from a human or animal subject (50) after ingestion of deuterated water is determined. The deuterium-resolved mass spectrum to determine a presence or amount of at least one deuterated metabolite. Also disclosed is a corresponding mass spectrometry system.

### TECHNICAL FIELD

The present invention relates to a mass spectrometry method and to a mass spectrometry system.

### PRIOR ART

The elucidation of biochemical and metabolic pathways is key for a deeper understanding of biological systems. Stable isotopes such as deuterium (²H) are powerful tools to achieve this endeavor.^{1,2} Deuterium (²H) has been deployed as a metabolic tracer in combination with different mass spectrometric methods such as hydrogen/deuterium exchange (HDX) mass spectrometry (MS) to explore dynamic protein structure or protein interactions and to study fatty acid/lipid biosynthesis.³⁻⁸

Recently, it has been suggested to monitor the incorporation of ²H into metabolites after ingestion of heavy water (²H₂O). As an example, X. Fu et al., “Measurement of lipogenic flux by deuterium resolved mass spectrometry”, Nature Communications 2021, 12:3756, doi: 10.1038/s41467-021-23958-4 discloses investigations of de novo lipogenesis by monitoring deuterium incorporation into fatty acids following the administration of deuterated water to mice. Blood samples were taken from the mice, and the blood plasma was treated as follows: Plasma samples (5 μL) were treated with 2 mL (1:1, v/v) methanol/dichloromethane (DCM) to precipitate proteins and extract triglycerides. Samples were vortexed for 1 min and then centrifuged for 5 min at 1635×g. The DCM layer was transferred to a new tube and dried under N₂. Samples were saponified with 1 mL 0.5 M KOH in methanol at 80° C. for 1 hr. Resulting FAs were extracted with 2 mL DCM/water (1:1, v/v) for 1 min, and the solvent was evaporated under N₂. Dried lipid extract was resuspended in 50 μL of 1% triethylamine/acetone and reacted with 50 μL of 1% PFBBr/acetone for 30 min at room temperature. To this solution, 1 mL of isooctane was added before MS analysis. Additionally, liver extract was treated by a similar method. The treated plasma or liver extract was then analyzed by deuterium-resolved mass spectrometry. This method requires an invasive procedure for obtaining the blood samples or the liver abstract. Furthermore, sample preparation is very complex and time-consuming.

EP3559675A1 discloses methods of monitoring amino acid levels by investigating exhaled breath condensate using LC-MS or LC-MS/MS methods. Deuterated leucine was used as an internal standard.

US 2010255598A1 discloses the detection of ²H-labelled markers in exhaled breath by FTIR spectroscopy. In one embodiment, a parent therapeutic agent is labeled with a ²H-labelled marker. Upon metabolism (e.g., via enzymatic action) of the therapeutic agent, the marker becomes volatile or semi-volatile and is present in the breath. In another embodiment, the therapeutic agent is associated with a ²H-labelled taggant, which in turn will generate a marker in the breath that is easily measurable. ²H-labelled therapeutic agents are difficult to obtain and can be extremely expensive because they need to be specifically synthesized.

L. Shi et al., “Optical imaging of metabolic dynamics in animals”, Nature Communications, 2018, 9(1):2995 combined deuterium oxide (²H₂O) probing with stimulated Raman scattering microscopy to image in situ metabolic activities.

S. Davies et al., “Rapid measurement of deuterium content of breath following oral ingestion to determine body water”, Physiol. Meas. 2001 November; 22(4):651-9, doi: 10.1088/0967-3334/22/4/301 discloses the use of flowing afterglow mass spectrometry to determine the deuterium content in exhaled breath water after oral ingestion of deuterated water. Only the deuterium content in the water signal is analyzed. This is also true for the following publications: P. Spanel et al., “Coordinated FA-MS and SIFT-MS analyses of breath following ingestion of D₂O and ethanol: total body water, dispersal kinetics and ethanol metabolism”, Physiol. Meas. 2005 August; 26(4):447-57, doi: 10.1088/0967-3334/26/4/011; C. Chan et al., “A non-invasive, on-line deuterium dilution technique for the measurement of total body water in haemodialysis patients”, Nephrol. Dial. Transplant. 2008 June; 23(6):2064-70, doi: 10.1093/ndt/gfn045; D. Smith et al., “Comparative measurements of total body water in healthy volunteers by online breath deuterium measurement and other near-subject methods”, Am. J. Clin. Nutr. 2002 December; 76(6):1295-301, doi: 10.1093/ajcn/76.6.1295

### SUMMARY OF THE INVENTION

It is an object of the present invention to provide a non-invasive method of investigating metabolic pathways in a subject, the method being capable to be carried out in real time and not requiring complex sample preparation protocols nor synthesis of labeled therapeutic agents.

This object is achieved by a method according to claim 1. Further embodiments of the invention are laid down in the dependent claims.

A mass spectrometry method is provided, comprising:
obtaining a gas sample comprising volatile metabolites that have been excreted from a human or animal subject after ingestion of deuterated water;
determining a deuterium-resolved mass spectrum of the gas sample; and
processing the deuterium-resolved mass spectrum to determine a presence or amount of at least one deuterated metabolite in the gas sample.

According to the present invention, a living human or animal subject ingests deuterated water. During or after a period of time of ingestion of the deuterated water, a gas sample is obtained from the subject, and a deuterium-resolved mass spectrum of the gas sample is determined. A deuterium-resolved mass spectrum is a mass spectrum that has sufficient resolution to distinguish between a deuterated compound and an isotopologue of the compound that has the same mass number but comprises no deuterons or a lower number of deuterons, instead comprising at least one heavy isotope of carbon (¹³C), nitrogen (¹⁵N) or oxygen (¹⁷O and ¹⁸O) in natural abundance. The deuterium-resolved mass spectrum is processed to determine a presence or amount of at least one deuterated metabolite in the gas sample. Specifically, the deuterium-resolved mass spectrum may be processed to identify at least one spectral feature associated with the presence of at least one deuterated metabolite in the gas sample, and an intensity of the spectral feature may be calculated to determine an indicator for the amount of the deuterated metabolite.

With this method, valuable insights into metabolic pathways in a subject can be gained. The method can readily be carried out in real time, i.e., the deuterium-resolved mass spectrum may be determined in real time immediately after the gas sample has been obtained from the subject, avoiding possible deterioration of the sample due to storage. The method does not require any complex sample preparation, as it would be the case with blood samples.

No isotopically labeled therapeutic agents are required. Therefore, the method can be carried out at relatively low cost.

In preferred embodiments, the gas sample comprises breath that has been exhaled by the subject. Breath samples can be particularly easily obtained from humans, and a breath sample can be readily transferred to an ion source in real time. In addition or in the alternative, the gas sample may comprise volatile metabolites that have been excreted through the skin.

Processing the deuterium-resolved mass spectrum may comprise a spectral separation step for separating a spectral feature associated with the deuterated metabolite from a spectral feature associated with an isotopologue of the metabolite that has identical mass number but comprises no deuterons or a lower number of deuterons. Such isotopologues will unavoidably be present due to the presence of heavy isotopes of carbon (¹³C), nitrogen (¹⁵N) and oxygen (¹⁷O and ¹⁸O) in natural abundance and should be distinguished from the deuterated metabolite of interest. The spectral separation step may comprise a binning procedure using a Kernel density function, as it is known in the art of high-resolution mass spectrometry per se. The Kernel density function may have a bandwidth that matches the resolution of the mass analyzer that was used for obtaining the mass spectrum.

Processing the deuterium-resolved mass spectrum may comprise determining an isotope ratio of the deuterated metabolite and a reference isotopologue of the metabolite in the gas sample. The reference isotopologue may be a fully protonated isotopologue, i.e., an isotopologue in which all deuterons of the deuterated metabolite are replaced by protons.

The steps of obtaining a gas sample may be repeated at a plurality of different times relative to ingestion of the deuterated water by the subject, and deuterium-resolved mass spectra may be determined for these gas samples to obtain a time series of the amount of the deuterated metabolite, in particular, of the aforementioned isotope ratio.

In another aspect, the present invention provides a mass spectrometry system that is configured for carrying out the method of the present invention. The mass spectrometry system comprises:
a mass spectrometer configured to receive a gas sample comprising volatile species that have been excreted from a subject after ingestion of deuterated water, the mass spectrometer comprising an ion source for ionizing at least a portion of the gas sample and a mass analyzer for determining a deuterium-resolved mass spectrum of the ionized gas sample; and
a data processing system configured to process the deuterium-resolved mass spectrum to identify and/or quantify at least one spectral feature associated with the presence of at least one deuterated metabolite in the gas sample.

In advantageous embodiments, the ion source is a SESI source, and wherein the mass analyzer is an Orbitrap-type mass analyzer. However, other types of ion sources and high-resolution mass analyzers may be used.

In some embodiments, the mass spectrometry system may be specifically configured to process breath samples in real time. To this end, the system may comprise an interface for coupling a breathing mask or a mouthpiece to the mass spectrometry system. In this manner it becomes possible to sample air that has been exhaled by the subject, the exhaled air forming the gas sample and to transfer the gas sample to the ion source in real time. The mass spectrometry system may further comprise said breathing mask or mouthpiece. In other embodiments, the gas sample may first be stored before the mass spectrum of the gas sample is determined, i.e., the gas sample may be analyzed offline. For instance, the gas sample may be collected by having a subject exhale into a bag and may be stored in the bag for off-line analysis, as it is commonly done in the well-known urea breath test for identifying infections by Helicobacter pylori. The mass spectrometry system may accordingly comprise an interface configured to connect to a breath test bag.

The data processing system of the mass spectrometry system may be configured to carry out any of the data processing procedures discussed above. To this end, the data processing system may comprise a computer processor and program memory storing program data that cause the computer processor to carry out the data processing procedures. The data processing system may in particular be an appropriately programmed general-purpose computer.

### BRIEF DESCRIPTION OF THE DRAWINGS

Preferred embodiments of the invention are described in the following with reference to the drawings, which are for the purpose of illustrating the present preferred embodiments of the invention and not for the purpose of limiting the same. In the drawings,

FIG. 1 shows a schematic diagram of an experimental setup for monitoring ²H-incorporation into metabolites in mice in real-time and in vivo;

FIGS. 2A-2C show portions of mass spectra for three exemplary metabolites;

FIGS. 2D-2F show time series of the ²H/¹H isotope ratio for the metabolites in FIGS. 2A-2C;

FIG. 3 shows a schematic diagram of an experimental setup for monitoring ²H-incorporation into metabolites in a human subject in vivo and in real time;

FIG. 4 shows a flow diagram of a mass spectrometry method;

FIGS. 5A-5C show time series of isotope ratios for selected metabolites in breath exhaled by a human subject.

### DESCRIPTION OF PREFERRED EMBODIMENTS

### Definitions

Isotopologue: Isotopologues are molecules that differ only in their isotopic composition. They have the same chemical formula and bonding arrangement of atoms, but at least one atom has a different number of neutrons than the parent.

Deuterated water: The term “deuterated water” is to be understood to comprise both “heavy water” (²H₂O) with two deuterium isotopes of hydrogen per molecules and “semi-heavy water” with one deuterium isotope per molecule (²H¹HO) at an abundance that is higher than the natural abundance.

Metabolite: The term “metabolite” is to be understood in the usual manner as any intermediate or end-product of metabolism. The term is to be understood to include both primary and secondary metabolites. Water is not considered a metabolite.

Deuterated metabolite: A deuterated metabolite is a metabolite in which at least one proton has been replaced by a deuteron. Deuterated water is not considered a deuterated metabolite.

Mass spectrometer: A mass spectrometer comprises an ion source (ionizer) and a mass analyzer. The ion source converts a portion of the sample into ions. An extraction system removes ions from the sample, which are then targeted to the mass analyzer.

SESI: Secondary electrospray ionization (SESI) is a spray type, ambient ionization method where ions are produced by means of an electrospray. These ions then charge vapor molecules in the gas phase when colliding with them. SESI is an efficient ambient ionization method, that, when combined with high-resolution mass spectrometry (SESI-HRMS), enables capturing fine isotopic structures.

Orbitrap-type mass analyzer: An Orbitrap is an ion trap mass analyzer consisting of an outer barrel-like electrode and a coaxial inner spindle-like electrode that traps ions in an orbital motion around the spindle. The image current from the trapped ions is detected and converted to a mass spectrum using the Fourier transform of the frequency signal.

Deuterium-resolved mass spectrum: A deuterium-resolved mass spectrum is a mass spectrum with sufficient resolution to distinguish spectral features (contributions to the spectral intensity) originating from a deuterated molecule and an isotopologue of the same molecule that has the same total mass number (i.e., the same molecular mass in Daltons when rounded to the next integer), but comprises a lower number of deuterons while instead comprising a heavy isotope of carbon, nitrogen or oxygen, i.e., a nucleus selected from ¹³C, ¹⁵N, ¹⁷O or ¹⁸O. These heavy isotopes are typically present at low natural abundance in organic molecules. A deuterium-resolved mass spectrum may be obtained by using a mass analyzer with sufficiently high resolving power. Preferably the resolving power is at least 120,000 at m/z 200. Such high resolving power can be achieved, e.g., with commercially available Orbitrap-type mass analyzers and will lead to well-separated spectral lines for the different isotopologues. However, a separation of spectral features arising from different isotopologues with identical total mass number may also be possible at lower resolving power, using spectral separation methods such as kernel density estimation. It should be kept in mind that the resolving power of Orbitrap-type mass analyzers scales with 1/sqrt (m/Z). Therefore it is easier to separate features from different isotopologues with identical total mass number for lighter molecules than it is for heavy molecules.

Isotope ratio: The term “isotope ratio” or “isotopic ratio” is to understood to designate the ratio of the concentration of a particular isotopologue in a sample and the concentration of another isotopologue of the same metabolite in the same sample. The isotope ratio is well approximated by the ratio of the intensities of the associated spectral features (“peaks”) in the mass spectrum and may therefore be equated to this ratio.

Breathing mask: A breathing mask is a mask that covers the mouth, and usually other parts of the face or head, designed to direct the wearer's breath to a particular apparatus.

### Example 1: Analysis of Excreted Gases from Mice

### Methods

Two healthy female specific-pathogen-free (SPF) C57BL/6 mice weighing approximately 18-22 g were used in an example. Mice were purchased from Guangdong Medical Laboratory Animal Center (Foshan, China) and housed under SPF conditions at 22±2° C. on a 12 h light/dark cycle, and fed using standard mice chow and water ad libitum. The mice were acclimatized for a week before the experiment began. The animal studies were approved by the Laboratory Animal Ethics Committee of Jinan University, and conducted in uniformity with national guidelines of the care and use of laboratory animals.

The deuterium oxide (²H₂O, 99.9 atom % ²H) was purchased from Merck (Darmstadt, Germany).

### SESI-HRMS Analysis

On day zero (D0) i.e. one day before the treatment with ²H₂O, both mice were fed with standard drinking water (H₂O) and a baseline measurement was acquired for both mice. Afterwards, mice were randomly divided into two groups. The treated group was fed with ²H₂O (80%, v/v); the control group was fed with H₂O. Volatile species emitted by the mice were measured at days 1, 2, 3, 10, 12 and 13 for 20 min by SESI-HRMS. SESI-HRMS analysis of mice's volatilome followed the procedure as previously reported¹³⁻¹⁶.

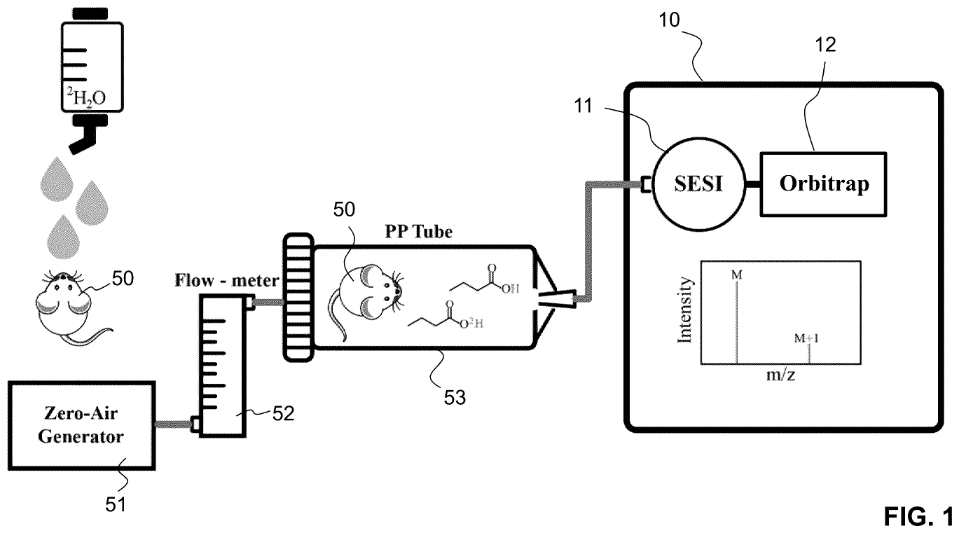

FIG. 1 shows a schematic diagram of an experimental setup for real-time monitoring of ²H incorporation in mice. Mice 50 were fed with ²H₂O or H₂O respectively and placed in a polypropylene tube 53 pervaded with 1 L/min of medical grade air produced by a zero-air generator 51 (Beijing Anjiehua Co., Ltd; Beijing, China) and controlled by a flow-meter 52. Volatile species emitted by the mice via skin and/or breath were transported into a lab-made SESI source 11. The sample was ionized in the SESI source and directly analyzed by a high-resolution mass spectrometer (HRMS, Thermo Scientific Q-Exactive Orbitrap MS, Waltham, MA USA) without sample pretreatment and without chromatographic separation. MS measurements were performed in polarity-switch mode with spray voltage of 2.5 kV; ion transfer capillary temperature of 150° C. The mass range was m/z 50-750 and the resolution was set to 70,000 at m/z 200. S-lens RF level was 50 and microscan was set to 1.

### Data Analysis

Pre-Processing: Data analysis was performed using MATLAB (version 2020b, MathWorks Inc., USA). Raw mass spectra files were converted to mzXML via msConvert (Proteowizard).¹⁷ Positive and negative scans per file were filtered out. We then computed the average spectrum of 100 scans per file and the spectra were recalibrated using a polynomial fitting. After calibration, the mass accuracy across the entire mass range was within 2 ppm for both polarities. Subsequently, the spectra were centroided and binned using a Kernel density function, selecting its bandwidth to match the instrument resolution at each m/z. The final feature list was obtained by centroiding the resulting Kernel density function. The signal intensity for each feature was computed by summing the intensity of the peaks within their full width at half maximum. As a result, a data matrix of 14 samples (seven time points for mouse drinking H₂O and seven time points for mouse drinking ²H₂O) ×3476 features in positive mode and ×1407 features in negative mode was obtained.

Post-Processing: In a first filtering step, the number of features was further reduced by considering only the cases were the signal intensity was greater than zero in at least four out of the seven data points measured for the mouse drinking ²H₂O. Molecular formulae were assigned to the remaining 1350 positive and 1131 negative mode features based on the measured accurate mass within a tolerance of 2 ppm. Protonated (for positive mode features) and deprotonated species (for negative mode features) fulfilling “seven golden rules”¹⁸ with only C, H, N and O were considered. ²H/¹H isotopologue pairs were searched by identifying mass spectral features with a mass difference of Δ=n*1.0063 u (±0.0005 u), where n=1,2, . . . 10. The ratio of the signal intensities ²H/¹H was computed for both mice at all the time points. Of the resulting ²H/¹H time traces, only those showing an increasing trend for the mouse drinking ²H₂O vs. the control mouse were further considered (i.e. 60 isotopologue pairs). Moreover, to explore similarities across the different kinetics of such ratio time traces, we conducted hierarchical cluster analysis (average method; correlation distance). We then used the “lipids and non-lipids main chemical class” metabolite sets in MetaboAnalystR (version 3.0.3)¹⁹ to assign potential compound candidates to metabolites undergoing ²H-enrichment. Furthermore, compound hits were cross-referenced with HMDB and KEGG databases using “CrossReferencing” function of MetaboAnalystR.

### Results

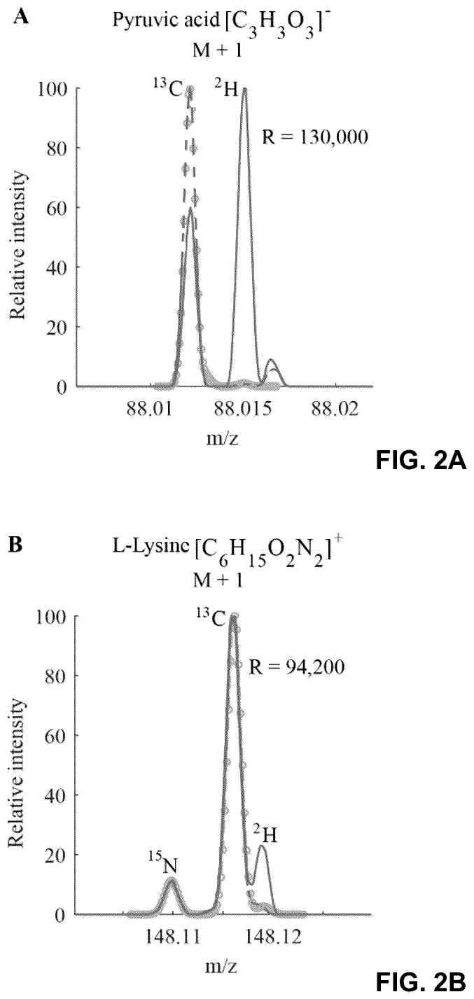

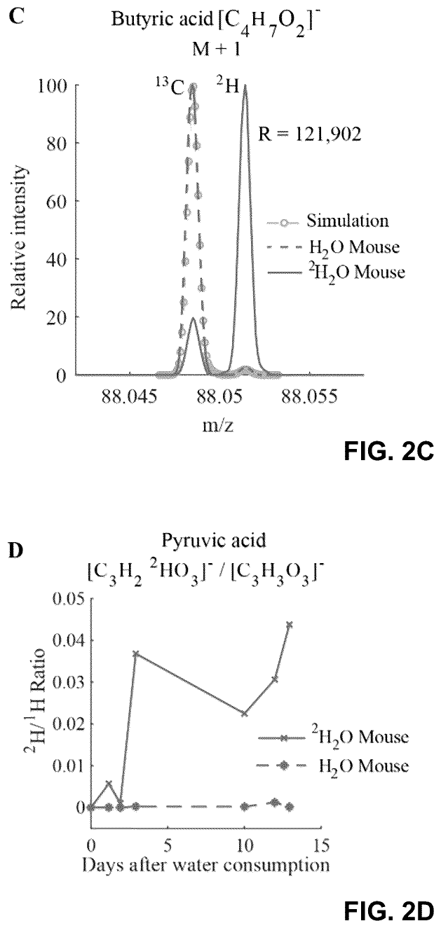

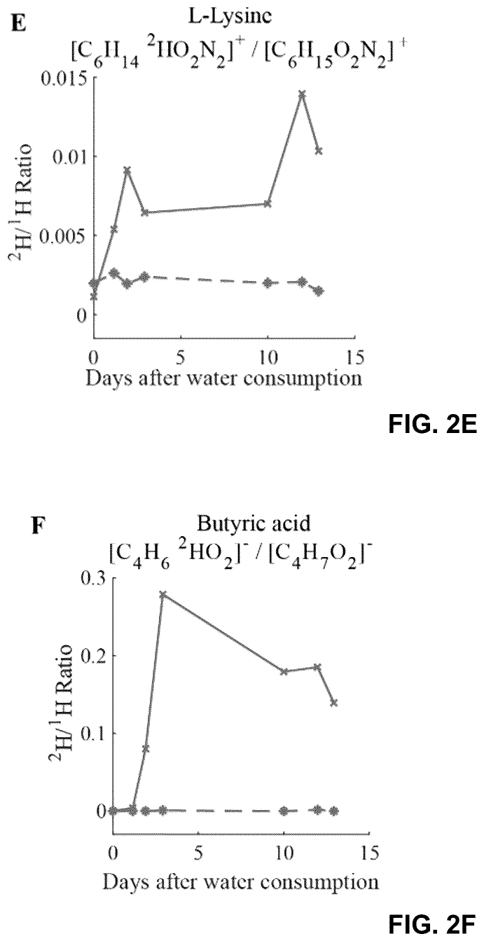

We identified 60 isotopologue ²H/¹H pairs (26 in positive and 34 in negative mode) fulfilling the mass difference and increasing intensity criteria. FIG. 2A shows an example of the M+1 isotopologue of [C₃H₃O₃]. It displays the experimental mass spectra for the mice drinking H₂O (broken line) and ²H₂O (solid line) at day 12, along with the simulated spectrum (circles) of [C₃H₄O₃−H]⁻ at a resolution of 130,000, which is matching the experimental resolution at m/z 88. This ion has been previously identified as pyruvic acid.^{20, 21} FIG. 2B and FIG. 2C illustrate two other examples in positive ion mode at m/z 148 and in negative ion mode at m/z 88.

Once we identified the species undergoing a relevant incorporation of ²H, we studied their evolution over time during the course of the experiment. FIGS. 2D-2F show three representative examples of ²H/¹H ratio time traces for the two mice investigated (i.e. ²H₂O and H₂O drinking mice) over 13 days. In most cases, the maximum ²H/¹H ratio occurred on day 12 after starting drinking ²H₂O. Despite most traces peaking on day 12, considerable differences in the kinetic profiles were observable by simple visual inspection (see FIGS. 2D-2F). To gain further insights into such different temporal patterns, we subjected the ratio time traces to hierarchical cluster analysis.

### Discussion

This example demonstrates that SESI-HRMS is a suitable tool to detect metabolic ²H-incorporation in vivo in real-time. The technique has the required sensitivity (i.e. part-per-trillion range²²) to enable the detection of species emitted under physiological conditions without any sample preconcentration, and at the same time renders the required mass resolution to enable the separation of fine isotopic structures. This is illustrated in FIG. 2A, which shows the M+1 mass spectrum of deprotonated pyruvic acid²¹ for the control mouse (broken line), the mouse drinking ²H₂O and the simulated spectrum at a resolution of ˜122,000 (circles), which is equivalent to the experimental one at this particular mass. Firstly, it shows a perfect match between the simulated spectrum and the experimental one from the mouse drinking H₂O, which provides confidence on the calibration procedure (within 2 ppm) and on the possibility to resolve ¹³C and ²H isotopic peaks having the same nominal mass number. Such perfect matching between the experimental and the simulated spectra—both at the m/z and the relative intensity domains—provides in addition further reassurance on the molecular formula assignments made for all ions. Another example is shown in FIG. 2B, whereby the most probable molecular formula at m/z 147.11268 was computed to be [C₆H₁₄O₂N₂+H]⁺, which has been previously identified in human breath as lysine²³. Clearly, the simulated spectrum and the experimental one (for H₂O mouse) match perfectly the m/z locations as well as the (relative) signal intensities for the natural-abundance ¹⁵N and ¹³C isotopologues. Even the natural-abundance ²H peak is partially resolved from the latter at a resolution of ˜94,000. Hence, the formula assignment is given with a high degree of confidence. FIG. 2C shows an additional example for the short chain fatty acid butyric acid²⁴. Many more M+1 mass spectra were sufficiently resolved to assign a molecular formula.

In all examples shown in FIGS. 2A-2C, there is a striking relative increase of the ²H peak for the mouse drinking heavy water, suggesting that monitoring non-invasively and in vivo the incorporation of ²H in metabolites after drinking ²H₂O during extended periods of time is feasible. This is illustrated in FIGS. 2D-2F, which show the ²H/¹H ratio time traces for the three species shown in FIGS. 2A-2C. By simple visual inspection, it becomes apparent that, as expected, the ratio remains constant and close to zero over the days for the control mouse drinking water. In contrast, for the mouse drinking ²H₂O, there is a match at day 0 (i.e. baseline), but on the following days, the ratio is clearly increased, departing away from the control mouse. Interestingly, we observed different dynamics for the ²H/¹H trajectories. For example, pyruvic acid spiked to nearly 4% on day three to then remain in that region until the end of the experiment (FIG. 2D). Lysine steadily increased until ˜1% on day 3 to then peak on day 12 at nearly 1.5% (FIG. 2E). In contrast, butyric acid remained at the natural abundance level until day one, to then raise on days two and three up to nearly 30%. It then declined during the subsequent days down to around 13% (FIG. 2F). Interestingly, this was one of the four examples of SCFAs that we found to undergo a detectable exchange of two (out its seven) hydrogens. The kinetic profile of ²H₂/¹H for butyric acid resembles very much the one for ²H/¹H, however, its maximum ratio was around ten times lower (i.e. ˜3% instead of ˜30% at day three). We found a similar behavior for all SCFAs as well as for lactic acid. However, for the latter, the ratio trended towards a clear increase as the days passed by (i.e., accumulation effect). Such disparity in the ²H/¹H ratio scale and kinetics suggests different mechanisms for the ultimate incorporation of ²H in the detected metabolites. For example, the maximum ²H/¹H ratio was found to be nearly one for C₄H₂²H₄O₂/C₄H₆O₂. However, this was an outlier as the majority of the identified ²Hₙ/H ratios corresponded to n=1 and the median ratio was in the order of 3% (median=0.0296; interquartile range=0.0631). There are mainly two mechanisms explaining the occurrence of ²H/¹H exchange: enzymatic irreversible exchange or non-enzymatic reversible exchange.

In the latter, ²H₂O forms N—²H, O—²H and S—²H bonds which occurs faster and more often because ²H/¹H exchange is more prominent when the hydrogen is bound to a heteroatom as a consequence of the intrinsic electronegativity provided by the free electron pair whereas in an enzymatic exchange ²H₂O forms C—²H bonds which is slower and dependent on the number of enzymes available and produced by the organism^{9, 25 26, 27}. While all the molecular formulae identified in this study contain either O and/or N-atoms, it is unclear at this point whether the observed ²H-incorporations correspond to enzymatic, non-enzymatic or both types of reactions.

The cluster analysis revealed three major groups of patterns in these ratio time series. Cluster one contains pyruvic acid and lactic acid. The similarity in the temporal profiles with an increasing trend over the course of the days can be explained by anaerobic glycolysis in red blood cells, muscle cells or gut microbiome, whereby glucose produces pyruvate, which is then converted to lactate when supply of oxygen is limited^{28, 29}. Cluster two contains lysine, which interestingly clusters together with m/z 161.1284, which fits with the molecular formula of methyl-lysine. In this cluster and cluster number three we found the three short chain fatty acids (i.e. acetic, propionic acid and butyric acid). These are the main products of gut microbiome activity and their importance modulating other metabolic, endocrine and immune functions is becoming increasingly evident.³⁰

### Example 2: Breath Analysis of a Human Subject

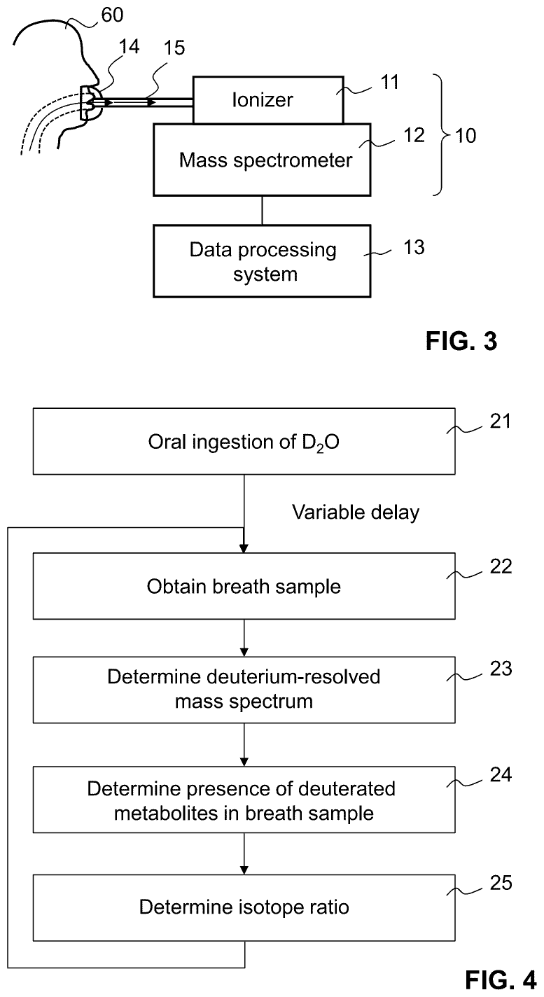

FIG. 3 schematically shows an experimental setup for monitoring ²H-incorporation into metabolites in a human subject in vivo and in real time. As in Example 1, a mass spectrometer 10 comprising a SESI source 11 and an Orbitrap high-resolution mass analyzer 12 may be used. The SESI source 11 may provide an interface for fluidically coupling a mouthpiece 14 to the SESI source 11 via tubing 15. In this manner, it becomes possible to analyze the exhaled breath from a human subject 60 in real time. The raw mass spectra obtained from the mass spectrometer 10 may be analyzed by a data processing system 13 comprising an appropriately programmed general-purpose computer.

A method of monitoring in vivo ²H-incorporation using this setup is schematically illustrated in the flow diagram of FIG. 4. In step 21, a human subject orally ingests deuterated water. A preferred amount is a single dose of 1 to 20 grams of 70% ²H₂O per kilogram of body water (where body-water is estimated to be 0.6×body mass in men and 0.5×body mass in women), or roughly 0.5 to 10 grams of 70% ²H₂O per kilogram of body mass. At these amounts, deuterated water is known not to have any toxic effects on the human body. In one example, a human subject (body mass 70 kg) ingested a single dose of 50 ml of ²H₂O (99.9%, v/v) within less than one minute, corresponding to approximately 1 g of 70% ²H₂O per kilogram of body mass. The subject exhaled into the mouthpiece at a plurality of different points in time after ingestion to obtain a plurality of breath samples (step 22), and deuterium-resolved mass spectra were determined for these breath samples in real time (step 23). The spectra were then analyzed for the presence of deuterated metabolites in the breath samples (step 24), and the isotope ratios of deuterated and protonated isotopologues of the same metabolites were determined for a plurality of metabolites (step 25).

Data processing was done in a very similar as described above for Example 1. Data processing comprised, in particular, the spectral separation of spectral features associated with deuterated metabolites and isotopologues having the same nominal mass number but a different number of deuterons. To this end, binning was performed by employing a Kernel density function having a bandwidth that matched the instrument resolution at each m/z, as in Example 1.

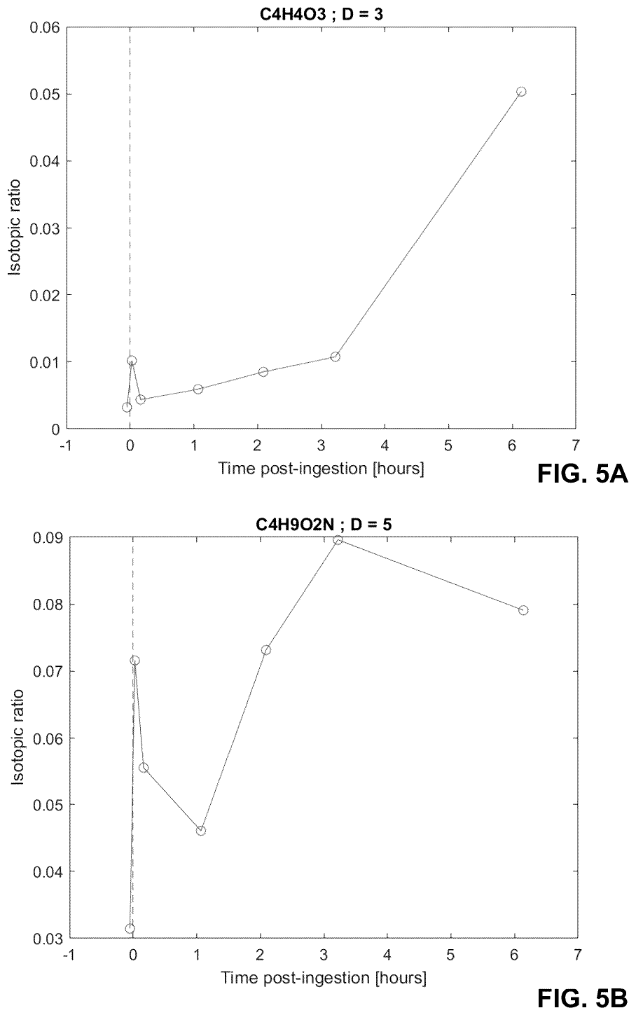

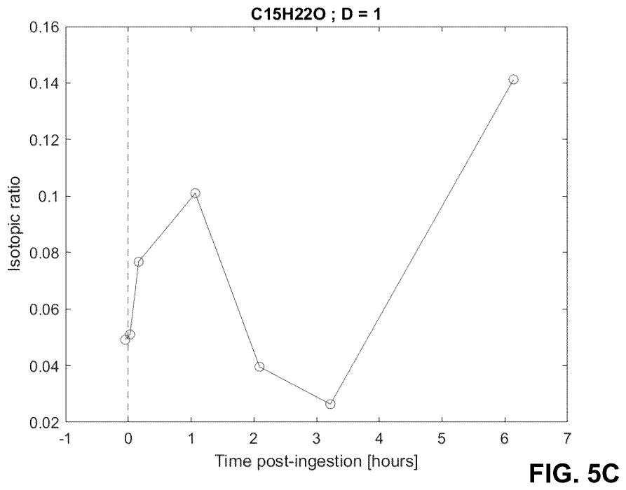

Several dozens of deuterated metabolites were identified and quantified by determining the isotope ratio relative to their fully protonated isotopologues. Selected time series of the measured isotope ratios are shown in FIGS. 5A-5C. A variety of different pattern were observed in these time series. For instance, a monotonic rise of isotope ratio was observed over the course of six hours after ingestions for some metabolites, as in FIG. 5A, while other metabolites exhibited more complex patterns, as illustrated in FIGS. 5B and 5C. The underlying mechanisms that cause these time series are the subject of ongoing investigations.

### CONCLUSIONS

SESI-HRMS allowed to monitor in vivo ²H-incorporation of metabolites in a non-invasive and real-time set-up, both for animals and humans, by sampling volatile compounds that are excreted via the skin or breath after oral ingestion of deuterated water. The obtained result show that the presently proposed method opens up new opportunities to use deuterium tracing to extend current metabolic studies, especially those with a focus on anaerobic glycolysis, lysine methylation and gut microbiome via monitoring of SCFAs.

### Modifications

In this study, secondary electrospray ionization was combined with high-resolution mass spectrometry (SESI-HRMS), using an Orbitrap-type mass analyzer. While this combination proved to be particularly well suited for the present purpose, the presently proposed method is not limited to a specific ionization method, and other types of ion sources may be employed, such as plasma ionization or atmospheric pressure chemical ionization. Neither is the method limited to a specific method of mass analysis, and other types of high-resolution mass analyzers may be used, such as FT-ICR mass analyzers.

The presently proposed method is not limited to a particular regime of administration of the deuterated water (e.g., single dose, multiple dose or continuous administration over a certain period of time) or to a particular scheme concerning the timing of when gas samples are obtained relative to the administration of the deuterated water. This is exemplified by the above Examples 1 and 2: In Example 1, the subject continuously ingested deuterated water over an extended period of time, and gas samples were obtained during this time. In Example 2, the subject ingested deuterated water only once over a very short period of time, and gas samples were obtained after the subject had stopped ingesting deuterated water. Many other schemes are conceivable, including schemes in which the ingestion of deuterated water follows a more complex pattern.

While in the above examples, mass analysis was carried out in real time, it is also possible to store the gas samples, e.g., in one or more bags, and to analyze them offline.

### REFERENCES

1. Schoenheimer, R. and D. Rittenberg, Deuterium as an Indicator in the Study of Intermediary Metabolism. Science, 1935. 82(2120): p. 156-7.
2. Miyagi, M. and T. Kasumov, Monitoring the synthesis of biomolecules using mass spectrometry. Philos Trans A Math Phys Eng Sci, 2016. 374(2079).
3. Vadas, O., et al., Using Hydrogen-Deuterium Exchange Mass Spectrometry to Examine Protein-Membrane Interactions. Methods Enzymol, 2017. 583: p. 143-172.
4. Lee, W.N., et al., In vivo measurement of fatty acids and cholesterol synthesis using D2O and mass isotopomer analysis. Am J Physiol, 1994. 266(5 Pt 1): p. E699-708.
5. Zhang, Z. and R.W. Vachet, Kinetics of Protein Complex Dissociation Studied by Hydrogen/Deuterium Exchange and Mass Spectrometry. Anal Chem, 2015. 87(23): p. 11777-83.
6. Miller, B.F., et al., CORP: The use of deuterated water for the measurement of protein synthesis. J Appl Physiol (1985), 2020. 128(5): p. 1163-1176.
7. Engen, J.R., et al., Developments in Hydrogen/Deuterium Exchange Mass Spectrometry. Anal Chem, 2021. 93(1): p. 567-582.
8. Fu, X.R., et al., Measurement of liipogenic flux by deuterium resolved mass spectrometry. Nature Communications, 2021. 12(1).
9. Shi, L., et al., Optical imaging of metabolic dynamics in animals. Nat Commun, 2018. 9(1): p. 2995.
10. Tejero Rioseras, A., et al., Comprehensive Real-Time Analysis of the Yeast Volatilome. Sci Rep, 2017. 7(1): p. 14236.
11. Singh, K.D., et al., Personalised therapeutic management of epileptic patients guided by pathway-driven breath metabolomics. Communications Medicine, 2021. 1(1): p. 21.
12. Shirasu, M. and K. Touhara, The scent of disease: volatile organic compounds of the human body related to disease and disorder. J Biochem, 2011. 150(3): p. 257-66.
13. Yin, Z., et al., In vivo monitoring of volatile metabolic trajectories enables rapid diagnosis of influenza A infection. Chem Commun (Camb), 2021. 57(39): p. 4791-4794.
14. Chen, X., et al., Online Real-Time Monitoring of Exhaled Breath Particles Reveals Unnoticed Transport of Nonvolatile Drugs from Blood to Breath. Analytical Chemistry, 2021. 93(12): p. 5005-5008.
15. Sinues, P.M.L., et al., Gauging circadian variation in ketamine metabolism by real-time breath analysis. Chemical Communications, 2017. 53(14): p. 2264-2267.
16. Li, X., et al., Drug Pharmacokinetics Determined by Real-Time Analysis of Mouse Breath. Angewandte Chemie-International Edition, 2015. 54(27): p. 7815-7818.
17. Kessner, D., et al., ProteoWizard: open source software for rapid proteomics tools development. Bioinformatics, 2008. 24(21): p. 2534-6.
18. Kind, T. and O. Fiehn, Seven Golden Rules for heuristic filtering of molecular formulas obtained by accurate mass spectrometry. BMC Bioinformatics, 2007. 8: p. 105.
19. Chong, J. and J. Xia, MetaboAnalystR: an R package for flexible and reproducible analysis of metabolomics data. Bioinformatics, 2018. 34(24): p. 4313-4314.
20. Martínez-Lozano, P. and J. Fernandez de la Mora, On-line Detection of Human Skin Vapors. Journal of the American Society for Mass Spectrometry, 2009. 20(6): p. 1060-1063.
21. Martinez-Lozano, P. and J. Fernandez de la Mora, Direct analysis of fatty acid vapors in breath by electrospray ionization and atmospheric pressure ionization-mass spectrometry. Analytical Chemistry 2008. 80(21): p. 8210-8215.
22. Martínez-Lozano, P., et al., Secondary Electrospray Ionization (SESI) of Ambient Vapors for Explosive Detection at Concentrations Below Parts Per Trillion. Journal of the American Society for Mass Spectrometry, 2009. 20(2): p. 287-294.
23. García-Gómez, D., et al., Real-Time Quantification of Amino Acids in the Exhalome by Secondary Electrospray Ionization-Mass Spectrometry: A Proof-of-Principle Study. Clinical Chemistry, 2016. 62(9): p. 1230-7.
24. Adam, M.E., et al., Mass-Spectrometry Analysis of Mixed-Breath, Isolated-Bronchial-Breath, and Gastric-Endoluminal-Air Volatile Fatty Acids in Esophagogastric Cancer. Anal Chem, 2019. 91(5): p. 3740-3746.
25. Kostyukevich, Y., et al., Hydrogen/deuterium exchange in mass spectrometry. Mass Spectrom Rev, 2018. 37(6): p. 811-853.
26. Grocholska, P. and R. Bachor, Trends in the Hydrogen-Deuterium Exchange at the Carbon Centers. Preparation of Internal Standards for Quantitative Analysis by LC-MS. Molecules, 2021. 26(10).
27. Englander, S.W., et al., Mechanisms and uses of hydrogen exchange. Curr Opin Struct Biol, 1996. 6(1): p. 18-23.
28. Bender, D.A., GLUCOSE|Function and Metabolism, in Encyclopedia of Food Sciences and Nutrition (Second Edition), B. Caballero, Editor. 2003, Academic Press: Oxford. p. 2904-2911.
29. Pessione, E., Lactic acid bacteria contribution to gut microbiota complexity: lights and shadows. Front Cell Infect Microbiol, 2012. 2: p. 86.
30. Dalile, B., et al., The role of short-chain fatty acids in microbiota-gut-brain communication. Nat Rev Gastroenterol Hepatol, 2019. 16(8): p. 461-478.

1. A mass spectrometry method comprising:
obtaining a gas sample comprising volatile metabolites that have been excreted from a human or animal subject after ingestion of deuterated water;
determining a deuterium-resolved mass spectrum of the gas sample; and
processing the deuterium-resolved mass spectrum to determine a presence or amount of at least one deuterated metabolite in the gas sample.

2. The mass spectrometry method of claim 1, further comprising:
ingestion of the deuterated water by the subject before the gas sample is obtained from the subject.

3. The mass spectrometry method of claim 1, wherein the deuterium-resolved mass spectrum is determined in real time immediately after the gas sample has been obtained from the subject.

4. The mass spectrometry method of claim 1, wherein the gas sample comprises breath that has been exhaled by the subject.

5. The mass spectrometry method of any claim 1, wherein processing the deuterium-resolved mass spectrum comprises:
carrying out a spectral separation step for separating a spectral feature associated with the deuterated metabolite from a spectral feature associated with an isotopologue of the metabolite that has identical mass number but comprises no deuterons or a lower number of deuterons.

6. The mass spectrometry method of claim 1, wherein processing the deuterium-resolved mass spectrum comprises:
determining an isotope ratio of the deuterated metabolite and a reference isotopologue of the metabolite in the gas sample.

7. The mass spectrometry method of claim 1,
wherein deuterium-resolved mass spectra are determined for a plurality of gas samples obtained from the subject at a plurality of different times relative to ingestion of the deuterated water by the subject, and
wherein a time series of the amount of the deuterated metabolite is determined from the deuterium-resolved mass spectra.

8. A mass spectrometry system, comprising:
a mass spectrometer configured to receive a gas sample comprising volatile species that have been excreted from a subject after ingestion of deuterated water, the mass spectrometer comprising an ion source for ionizing at least a portion of the gas sample and a mass analyzer for determining a deuterium-resolved mass spectrum of the ionized gas sample; and
a data processing system configured to process the deuterium-resolved mass spectrum to identify and/or quantify at least one spectral feature associated with the presence of at least one deuterated metabolite in the gas sample.

9. The mass spectrometry system of claim 8, wherein the ion source is a SESI source, and/or wherein the mass analyzer is an Orbitrap-type mass analyzer.

10. The mass spectrometry system of claim 8, comprising an interface for a breathing mask or a mouthpiece to enable transfer of air that has been exhaled by the subject to the ion source in real time.

11. The mass spectrometry system of claim 8, wherein the data processing system is configured to carry out a spectral separation for separating a spectral feature of the deuterated metabolite from a spectral feature of an isotopologue of the metabolite that has identical mass number but comprises no deuterons or a lower number of deuterons.

12. The mass spectrometry system of claim 8, wherein the data processing system is configured to determine an isotope ratio of the deuterated metabolite and a reference isotopologue of the metabolite in the gas sample.

13. The mass spectrometry system of claim 8, wherein the data processing system is configured to determine a time series of the amount of the deuterated metabolite, based on a plurality of deuterium-resolved mass spectra of samples obtained from the subject at a plurality of different times relative to ingestion of the deuterated water by the subject.

14. The mass spectrometry method of claim 5, wherein the spectral separation is carried out using a Kernel density function.

15. The mass spectrometry system of claim 11, wherein the the data processing system is configured to carry out the spectral separation using a Kernel density function.
